(19) United States
(12) Patent Application Publication
(10) Pub. No.: US 2009/0005601 A1
(43) Pub. Date: Jan. 1, 2009

(54) Process for preparing low malodor dimethyl sulfoxide

(75) Inventors: George Kvakovszky, Slidell, LA (US); David Villarrubia, II, Charles City, LA (US); Scott Stevenson, Lake Jackson, TX (US); Charles G. Ashe, Carriere, MS (US)

Correspondence Address: MERCHANT & GOULD PC, P.O. BOX 2903, MINNEAPOLIS, MN, 55402-0903, US

(73) Assignee: Gaylord Chemical Company LLC

(21) Appl. No.: 11/901,322

(22) Filed: Sep. 17, 2007

Related U.S. Application Data
(60) Provisional application No. 60/937,680, filed on Jun. 29, 2007.

(51) Int. Cl. C07C 315/06

(52) U.S. Cl. 568/27; 422/5

(57) ABSTRACT

The disclosed process relates to the removal of malodorous compounds from dimethyl sulfoxide.

### CROSS REFERENCE TO RELATED APPLICATIONS

This application claims priority to the U.S. Provisional Application Ser. No. 60/937,680, filed Jun. 29, 2007, the disclosure of which application is hereby incorporated in its entirety by this reference.

### FIELD OF THE DISCLOSURE

The disclosed process relates to the removal of volatile organic compounds from dimethyl sulfoxide, especially malodorous organic compounds, inter alia, dimethyl sulfide.

### BACKGROUND

Dimethyl sulfoxide (DMSO) is an important polar aprotic solvent and has been in commercial use since 1953. It can be manufactured commercially by reacting the black liquor from digestion in the Kraft pulp process, with molten sulfur to form dimethyl sulfide which is then oxidized with nitrogen tetroxide. DMSO is also very commonly used in a wide variety of end use applications as a polar aprotic solvent. DMSO is less toxic than other common polar aprotic solvents such as dimethylformamide, dimethylacetamide, N-methyl-2-pyrrolidone, and HMPA. DMSO can also be an effective component of paint stripper compositions being much safer than many of the others such as nitromethane and dichloromethane (See U.S. Pat. No. 6,673,157).

DMSO has also found use in the medical and pharmaceutical fields. The low toxicity and high skin permeability of DMSO have led to extensive studies in numerous biological systems, including humans. Inorganic salts or small-molecular-weight organic compounds dissolved in DMSO can be transported across skin membrane. For example, DMSO has been studied at different concentrations in the treatment of urinary bladder damage in patients with cervix uterine cancer (CUC) (Nelkasova, N. et al., Vestn Rentgenol Radiol. (2006) May-June; (3):47-51).

DMSO is manufactured from the oxidation of dimethyl sulfide, and incomplete oxidation or other side reactions can produce traces of various malodorous sulfur-containing compounds. Moreover, DMSO does in fact undergo slow but potentially significant decompositon reactions when heated for significant periods or distilled, and the resulting malodorous impurities are not and cannot typically be completely removed by the distillation processes that are typically used to purify most common grades of commercially available DMSO, as reflected by the fact that most individuals who work with DMSO know it typically has a slight sulfurous odor.

Although melt crystallization can produce extremely pure DMSO having little odor (see U.S. Pat. No. 6,414,194), melt crystallization is, nevertheless, an expensive purification process that can dramatically increase the cost of commercially available phamaceutical grade DMSO. Therefore, despite the availability of high cost melt crystallized DMSO, many of the more common uses of DMSO as a solvent cannot bear the expense of using melt crystallized DMSO, and do suffer from problems with the odor of DMSO. Therefore, there is a long felt need for an inexpensive process for preparing low malodor dimethyl sulfoxide.

### SUMMARY

The present disclosure relates to a process for deodorizing liquid dimethyl sulfoxide (DMSO) that encompasses contacting liquid DMSO with a gas thereby reducing the amount of malodorous compounds present by at least about 50%.

### BRIEF DESCRIPTION OF THE DRAWINGS

FIG. 1 depicts a graph of the decrease in the concentration of dimethyl sulfide over time for a sample of dimethyl sulfoxide treated by the disclosed process at room temperature.

FIG. 2 depicts a calibration curve for the determination of dimethyl sulfide by gas chromatography.

FIG. 3 depicts the amount of dimethyl sulfide present during the deodorization of DMSO as described in Example 1.

FIG. 4 depicts a graph of the decrease in the concentration of dimethyl sulfide over time for a sample of dimethyl sulfoxide treated by the disclosed process at 42.4° C.

FIG. 5 depicts various configurations of on line spargers according to disclosed process: A) Elbow Mounted, B) Side Mounted, C) Tee Mounted, D) Sanitary Tee Mounted, E) Side Angle Mounted.

### DETAILED DESCRIPTION

The disclosed process solves the unmet problem of providing a method for the low cost production of large quantities of DMSO having a reduced amount of volatile organic compounds, especially volatile organic compounds that have an objectionable odor or when left in the liquid DMSO can form volatile organic compounds having an objectionable odor. The disclosed process in one embodiment can provide low malodorous or deodorized DMSO.

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Deodorizing” means the removal of unwanted compounds that have an undesirable odor.

“Introducing” means to pass a flow (i.e., as in a stream) of a non-reactive gas as described herein into the liquid dimethyl sulfoxide.

“Gas” is any substance that is in the gaseous state of matter within the temperature range and under the conditions of the disclosed process. Gases include nitrogen, helium, argon, neon, air, oxygen, carbon dioxide, carbon monoxide, C₁-C₅ alkanes, for example, methane, ethane, propane, butane, iso-butane, pentane, and isopentane, water vapor, and the like. “Non-reactive gas” or “gas non-reactive with” means a gas that in general does not react with dimethyl sulfoxide or acts as an inert gas within the temperature range and other conditions of the disclosed process. Non-reactive gases include “inert” gases such as nitrogen, helium, argon, neon, and the like.

“Time sufficient” means a length of time necessary to achieve the desired result, for example, to lower the amount of malodorous compounds present in dimethyl sulfoxide to an amount not detectable by a human or by an analytical method.

“Volatile organic compounds” include any compounds having sufficient volatility such that they are removed from liquid DMSO under the conditions of the disclosed process. Volatile organic compounds include “malodorous compounds” which means a compound that has an aesthetically undesirable odor. Among malodorous compounds typically found in non-deodorized and commercially available dimethyl sulfoxide are volatile sulfur containing compounds such as hydrogen sulfide (H₂S), dimethyl sulfide ((CH₃)₂S), crotyl mercaptan (CH₃CH═CHCH₂SH), methyl mercaptan ethyl (CH₃SH), mercaptan (CH₃CH₂SH), ethyl methyl sulfide (CH₃CH₂SCH₃), dimethyl disulfide (CH₃SSCH₃), and bis(methylthio)methane (CH₃SCH₂SCH₃). In addition, DMSO that has been used in a chemical synthesis and which is being recycled for re-use can comprise volatile organic compounds. These volatile organic compounds can be solvents or by products of the original chemical synthesis that can be removed by the process disclosed herein.

“Undetectable” as used, for example, refers to an amount of dimethyl sulfide or other volatile organic compound present at a level that is undetectable by standard analytical techniques, inter alia, gas chromatography, gas chromatography coupled with mass spectroscopy, and the like. As such, the instrument does not indicate the presence of a compound although an amount of dimethyl sulfide or volatile organic compound may be present. Stated in another way, the amount of dimethyl sulfide or volatile organic compound is present in an amount beyond the limit that is detectable. Undetectable also refers to the ability to perceive the presence of a compound, for example, dimethyl sulfide using the human sense of smell.

### Processes for Dimethyl Sulfoxide

The processes disclosed herein relate to inexpensive and commercially reasonable methods for treating DMSO with streams of gases, especially non-reactive gases, to produce liquid dimethyl sulfoxide having very low levels of volatile organic compound impurities. Many of the volatile organic compounds that can be removed by the disclosed process are malodorous compounds, such as, but not limited to dimethyl sulfide, methyl mercaptan, hydrogen sulfide, ethyl mercaptan, and the like. In one embodiment, the process comprises treating liquid dimethyl sulfoxide having one or more volatile organic compounds present with a gas that does not react with dimethyl sulfoxide under the conditions of disclosed processes. In a further embodiment, the process comprises treating liquid dimethyl sulfoxide having one or more undesirable malodorous compounds present with a gas that does not react with dimethyl sulfoxide under the conditions of disclosed processes.

In one embodiment, the process comprises (a) initially providing liquid dimethyl sulfoxide comprising one or more malodorous compounds for treatment, and (b) introducing into the liquid dimethyl sulfoxide a flow of one or more gases non-reactive with the dimethyl sulfoxide, so as to reduce the concentration of one or more of the malodorous compounds in the dimethyl sulfoxide by at least about 50% as compared to the concentration of the one or more malodorous compounds originally present in the liquid dimethyl sulfoxide before treatment.

In another embodiment, the process comprises (a) initially providing liquid dimethyl sulfoxide comprising one or more volatile organic compounds for treatment, and (b) introducing into the liquid dimethyl sulfoxide a flow of one or more gases non-reactive with the dimethyl sulfoxide, so as to reduce the concentration of one or more of the volatile organic compounds in the dimethyl sulfoxide by at least about 50% as compared to the concentration of the one or more volatile organic compounds originally present in the liquid dimethyl sulfoxide before treatment.

There are certain volatile organic compounds, especially odorous compounds such as methyl mercaptan or ethyl mercaptan whose concentration in DMSO may be below the threshold of the human nose. Nevertheless, such compounds may have an undesirable, deleterious, intensifying or synergistic effect which may augment the undesirable odor of other odorants, including but not limited to, dimethyl sulfide. Thus, certain compounds which may be present in concentrations below the threshold of the human nose, or below the threshold of detection by analytical instrumentation, may still have an undesirable effect on odor. The current processes are also useful for reducing or eliminating such compounds.

The processes disclosed herein are also useful for removing volatile organic compounds that are not considered to be highly malodorous, for example, methylene chloride, ethyl acetate, and hexane. The processes disclosed herein can be one step in a number of steps in an overall chemical process. For example, the disclosed process can be a part of a solvent recovery/recycle step. A process utilizing DMSO as a solvent can, during one or more process steps, be extracted with an organic solvent, inter alia, methylene chloride. The disclosed process is capable of removing the residual solvent from the DMSO as part of a solvent recovery/recycle process.

The processes described herein are useful for reducing or eliminating the mild but undesirable odors that are typically associated with most commercially available grades of dimethyl sulfoxide, wherein the odor is often perceptible by the human sense of smell. These undesirable odors can be detrimental to the use of dimethyl sulfoxide in many end use applications that involve human contact with the dimethyl sulfoxide.

In some embodiments, the disclosed processes described herein relate to processes for deodorizing dimethyl sulfoxide comprising
(a) initially providing liquid dimethyl sulfoxide comprising one or more malodorous compounds for treatment, and
(b) introducing into the liquid dimethyl sulfoxide a flow of one or more gases non-reactive with the dimethyl sulfoxide,
so as to reduce the concentration of one or more of the malodorous compounds in the dimethyl sulfoxide by at least about 50% as compared to the concentration of the one or more malodorous compounds originally present in the liquid dimethyl sulfoxide before treatment.

The processes described herein can be used to inexpensively remove volatile organic compounds from liquid dimethyl sulfoxide, for example, to deodorize liquid dimethyl sulfoxide, and may be used for improving the odor of formulations, mixtures or solutions comprising predominantly DMSO, either in a production facility for manufacturing dimethyl sulfoxide, or for deodorizing dimethyl sulfoxide after it has been purchased or is in use for the many varied end use applications of dimethyl sulfoxide. The processes disclosed herein are particularly useful for deodorizing commercially available grades of dimethyl sulfoxide. Such currently commercially available dimethyl sulfoxide is typically produced and/or purified by vacuum distillation, and can have high chemical purity, but typically contains small amount and/or traces of one or more malodorous impurities that impart the objectionable odors perceptible by humans. Dimethyl sulfoxide is generally perceived by those of ordinary skill in the art as a thermally stable compound and/or solvent. Surprisingly, those of ordinary skill in the art have apparently not recognized the practical relationship of the traces of malodorous compounds that are produced when dimethyl sulfoxide is heated or distilled, and the relationship of those traces of malodorous compounds to the perceived mildly objectionable characteristic odor of dimethyl sulfoxide. Also surprisingly, those of ordinary skill in the art have apparently not recognized that such objectionable malodorous compounds and/or odors can be removed by introducing a flow of a gas through liquid dimethyl sulfoxide.

In many embodiments of the processes described herein, the liquid dimethyl sulfoxide initially provided to and/or treated by the processes have an initial purity of greater than or equal to about 96% by weight, or greater than or equal to about 98% by weight, or greater than or equal to about 99% by weight, greater than or equal to about 99.5% by weight, or greater than or equal to about 99.8% by weight. In further embodiments of the processes disclosed herein, the liquid dimethyl sulfoxide can be provided initially as a formulation, mixture or solution comprising greater than or equal to about 50% by weight of DMSO. DMSO compositions comprising greater than 50% by weight of DMSO can be treated by the disclosed processed for odor reduction or removal of volatile organic compounds.

In the processes described herein, a gas, especially a gas that is non-reactive with the dimethyl sulfoxide, is introduced and/or dispersed into, or sparged into the liquid dimethyl sulfoxide, often in combination with mild heating and/or agitation, and flows through the liquid dimethyl sulfoxide in the form of bubbles, which absorb and/or sweep at least some of the volatile organic compounds into the gas, which then naturally rises and separates from the liquid dimethyl sulfoxide, effectively “sweeping” the impurities out of the liquid dimethyl sulfoxide and thereby deodorizing it.

Non-limiting examples of gases that can be used for the disclosed processes include nitrogen, argon, carbon dioxide, air, or mixtures of two or more of these gases. For example, dry nitrogen gas can be obtained in bulk and at a moderate cost and is capable of deodorizing or otherwise removing dimethyl sulfoxide under the herein disclosed conditions. Another example includes the use of argon gas. Air can be used, especially if the amount of air used is outside the flammability envelope, for example, when the formulator chooses lower temperature conditions and longer contact times for removal of the unwanted volatile organic compounds. However, the formulator can use “low oxygen air” that consists of mixtures of ambient air having about 21% oxygen and nitrogen to reduce the cost of nitrogen. For example an admixture of 75% nitrogen and 25% air is a suitable lower cost use of nitrogen gas as the non-reactive gas component of the disclosed process. Other admixtures of gases are also equally suitable for use. The formulator can use the non-reactive gas based upon the exact conditions of deodorization, or can contact the dimethyl sulfoxide with different non-reactive gases or mixtures thereof, during several discrete steps during the process.

The treatment with one or more non-reactive gases can be conducted by contacting the gas with the dimethyl sulfoxide in any manner suitable to the formulator or adaptable to the equipment that the formulator currently uses for preparing dimethyl sulfoxide. It is often desirable that the gases be highly dispersed so as to initially form small bubbles, which pass through and can sweep one or more of the malodorous impurities from the liquid dimethyl sulfoxide as they rise and/or coalesce, and/or eventually separate from the liquid dimethyl sulfoxide.

For example, a non-reactive gas can be introduced into the bottom of a vessel containing the dimethyl sulfoxide through one or more gas lines, a gas line equipped with a frit, or other sparging device, for example, a sintered glass or metal frit. The process disclosed herein can be adapted such that the non-reactive gas is introduced in a line through a frit or other type of gas introducing device so that the gas is introduced in the form of bubbles of the desired size, optional while the dimethyl sulfoxide is being pumped from one vessel to another. The disclosed process is therefore adaptable to both batch and continuous processes. The vessels are typically configured and operated in such a manner as to exclude atmospheric gases, and thereby exclude atmospheric moisture from the highly hygroscopic dimethyl sulfoxide.

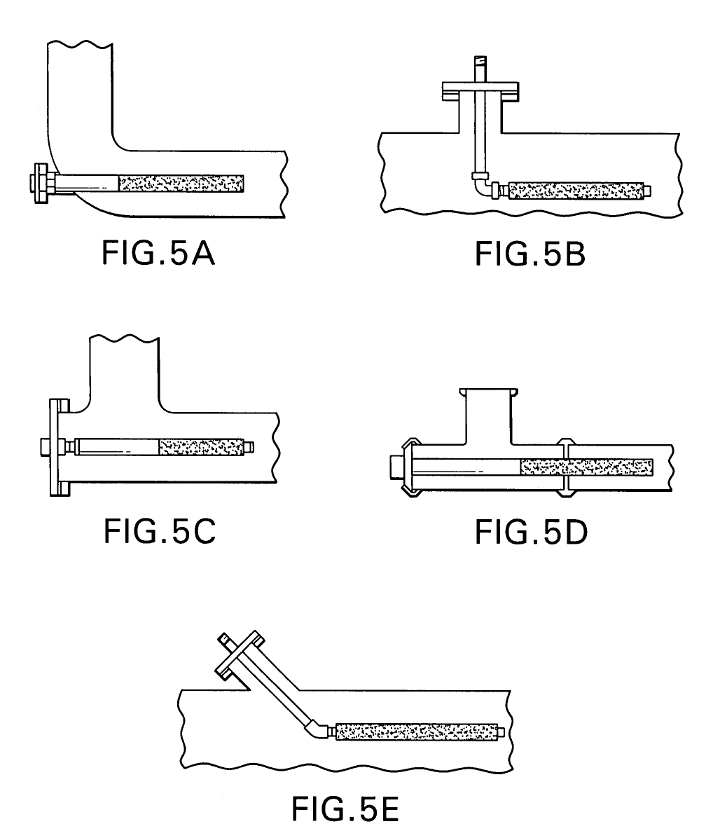

FIG. 5 depicts several types of pipe mounted intrusive sparger elements that can be used to introduce a non-reactive gas into the feed lines of storage tanks, temporary holding tanks or other vessels or containers of a process for deodorizing DMSO. In addition, non-intrusive (in-line) sparging can also be used to introduce a non-reactive gas. As it relates to the introduction of a non-reactive gas into a stream or vessel of DMSO comprising one or more malodorous compounds, the smaller the pore opening the larger the total surface area of the bubbles of gas that can be dispersed through a specific volume. Non-limiting examples of static and dynamic spargers useful for either batch or continuous processes for deodorizing DMSO include INCONEL™600, MONEL™400, Nickel 200, HASTELLOY™ C276, C22, and X, and Alloy 20 spargers available from Mott Corporation, Farmington, Conn.

The disclosed process can be conducted at any temperature above the melting point of dimethyl sulfoxide (about 18.6° C.) and below the boiling point (189° C.). Increasing the temperature of the liquid dimethyl sulfoxide permits faster removal of the undesirable malodorous impurities, but undesirably increased temperatures can also promote the slow thermal decomposition reactions of DMSO that can produce malodorous impurities, and the rate of the undesirable thermal decomposition reactions can depend on the purity of the initial DMSO. Accordingly, in the processes disclosed herein, the liquid DMSO is often typically mildly heated while being purified within controlled ranges (by any suitable means known to those of ordinary skill in the art, such as by external heating elements, or in some embodiments by heating the gas). One such process temperature range is from about 30° C. to about 100° C. A further example of a suitable process temperature range is from about 35° C. to about 80° C. A yet further example of a suitable process range is from about 40° C. to about 60° C. A still further example of a suitable process temperature range is from about 40° C. to about 50° C. The following are non-limiting examples of temperatures at which the disclosed process can be conducted; 41° C., 42° C., 43° C., 44° C., 45° C., 46° C., 47° C., 48° C., and 49° C. Also any fractional temperature thereof, for example, 42.1° C., 42.4° C., and 44.5° C. is suitable for use. In some embodiments of the disclosed process, the temperature can be raised or lowered during the course of the deodorization. For example, the process may begin at a first temperature, for example, 35° C. at which temperature the amount of malodorous compounds are greatly reduced, after which the temperature is raised slowly or all at once to a second temperature wherein the last remaining amounts of detectable malodorous compounds are removed.

The disclosed process can achieve a reduction in the amount of malodorous compounds present in DMSO such that the amount of malodorous compounds remaining in the DMSO is an amount less than or equal to about 50% of the original amount present. In another example, the amount of malodorous compounds remaining in the DMSO is an amount less than or equal to about 75% of the original amount present.

In a further example, the amount (i.e. concentration) of malodorous compounds remaining in the DMSO is an amount less than or equal to about 90% by weight of the original amount present. In a yet further example, the amount of malodorous compounds remaining in the DMSO is an amount less than or equal to about 95% of the original amount present. In a still further example, the amount of malodorous compounds remaining in the DMSO is an amount less than or equal to about 99% of the original amount present.

The disclosed process can remove the malodorous compounds present in DMSO such that the amount present is less than the odor detection threshold of a human. In another example, the disclosed process can remove the malodorous compounds present in DMSO such that the amount present is less than or equal to 2 ppm. In a further example, the disclosed process can remove the malodorous compounds present in DMSO such that the amount present is less than or equal to 1.5 ppm. In a yet further example, the disclosed process can remove the malodorous compounds present in DMSO such that the amount present is less than or equal to 1 ppm. In a still further example, the disclosed process can remove the malodorous compounds present in DMSO such that the amount of an undesirable volatile organic compound is undetectable by human olfactory perception (using the human sense of smell) or by an analytical instrument.

One example of the disclosed process includes a process comprising:
a) contacting liquid dimethyl sulfoxide containing dimethyl sulfide with a non-reactive gas; and
b) heating the liquid dimethyl sulfoxide while in contact with a non-reactive gas to a temperature of from about 30° C. to about 50° C.;
thereby forming dimethyl sulfoxide having an amount of dimethyl sulfide of less than or equal to about 2 ppm.

A further example of the disclosed process includes a process comprising:
a) contacting liquid dimethyl sulfoxide containing dimethyl sulfide with nitrogen gas; and
b) heating the liquid dimethyl sulfoxide while in contact with nitrogen gas to a temperature of from about 30° C. to about 50° C.;
thereby forming dimethyl sulfoxide having an amount of dimethyl sulfide of less than or equal to about 1.5 ppm.

A yet further example of the disclosed process includes a process comprising:
a) contacting liquid dimethyl sulfoxide containing dimethyl sulfide with nitrogen gas; and
b) heating the liquid dimethyl sulfoxide while in contact with nitrogen gas to a temperature of from about 30° C. to about 50° C.;
thereby forming dimethyl sulfoxide having an amount of dimethyl sulfide of less than or equal to about 1 ppm.

A still further example of the disclosed process includes a process comprising:
a) contacting liquid dimethyl sulfoxide containing dimethyl sulfide with a gas; and
b) heating the liquid dimethyl sulfoxide while in contact with a gas to a temperature of from about 30° C. to about 50° C.;
thereby forming dimethyl sulfoxide having an amount of dimethyl sulfide of less than or equal to about 2 ppm.

A yet still further example of the disclosed process includes a process comprising:
a) contacting liquid dimethyl sulfoxide containing on or more volatile organic compounds with a gas; and
b) heating the liquid dimethyl sulfoxide while in contact with a gas to a temperature of from about 30° C. to about 50° C.;
thereby forming dimethyl sulfoxide having an amount of volatile organic compounds of less than or equal to about 2 ppm.

The disclosed process can be conducted under reduced pressure in order to achieve the final results that are desired or to adapt the process to equipment available to the formulator.

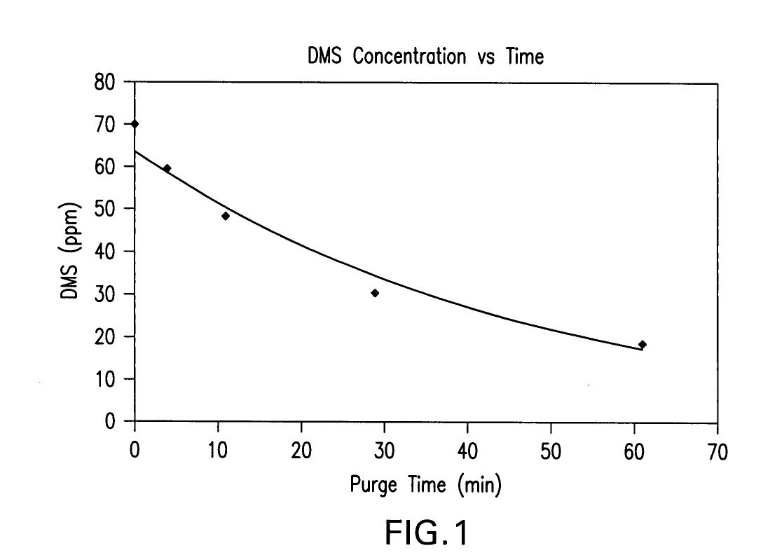

FIG. 1 depicts the amount of an impurity, dimethyl sulfide, present during an isothermal (42.4° C.) deodorization of dimethyl sulfoxide. After 60 minutes of contact with nitrogen gas the amount of dimethyl sulfide present has been reduced from approximately 69.9 ppm to 18.6 ppm (FIG. 1 indicates the actual data points from Table 1). Table 1 herein below presents the data points in tabular form for the graph appearing in FIG. 1.

TABLE 1

| Time (min) | DMS (ppm) |
|---|---|
| 0 | 69.9 |
| 4 | 59.3 |
| 11 | 48.2 |
| 29 | 30.3 |
| 61 | 18.6 |

The data of Table I is plotted in FIG. 1 with an exponential curve fit using the equation derived from this trendline, the formulator can extrapolate or interpolate to the amount of time necessary at a given temperature to obtain a desired final concentration of a malodorous organic compound.

### Analytical Procedure

Because the disclosed process can remove dimethyl sulfide and other malodorous impurities to a level below the odor detection threshold, analytical methods have been adapted to compensate for several factors. One factor is the decomposition of DMSO to dimethyl sulfide during the analysis processes due to the heat of the injection port of commercially available gas chromatographs.

To establish a reproducible and consistent method for determining the level of dimethyl sulfide present in analyzed samples of DMSO, a standardized calibration curve, in conjunction with the disclosed standardized method, can be constructed such that it allows for both direct measurement of dimethyl sulfide content, as well as extrapolation or interpolation of the amount of dimethyl sulfide present.

The standardization curve was produced by serial dilution of high purity, melt crystallized DMSO as the dilution solvent. The DMSO solvent was vigorously nitrogen sparged prior to use. A stock solution of 10,000 ppm dimethyl sulfide, DMS (99.53% pure) was prepared by spiking 50 mL of the above pure, melt crystallized, DMSO with 502 microliters of 99.53% pure dimethyl sulfide (DMS). After two serial dilutions, a working standard of 10 ppm DMS in DMSO was obtained. The 10 ppm working standard was further diluted yielding five calibration standards ranging from 0.1 to 1.0 ppm DMS in DMSO. An Agilent Technologies 6890N GC (gas chromatographic instrument) equipped with an Agilent Technologies 7683 series auto injector and a Quadrex 007, 15 meter×0.32 mm internal diameter, 3 micrometer film thickness column was employed for all analyses. Standards were analyzed under isobaric (5 psi) and isothermal (100° C.) conditions.

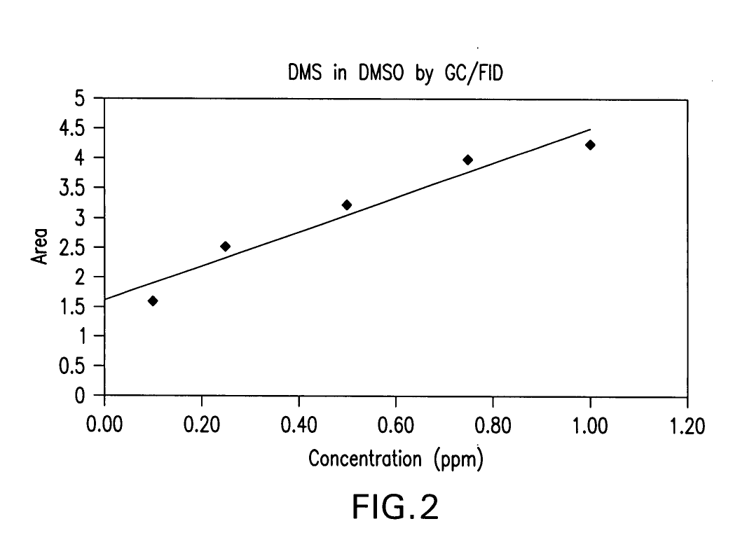

FIG. 2 shows the resulting calibration curve for this procedure. Using this calibration curve withdrawn samples can be analyzed for the content of dimethyl sulfide. The resultant area counts of each standard from the Flame Ionization Detector (FID) were plotted against the absolute concentration of dimethyl sulfide (DMS) in ppm for each standard. A linear least squares fit of the data was generated with an acceptable correlation coefficient of 0.9445. This confirms a direct linear correlation between absolute sample concentration in ppm and gas chromatograph area counts.

As discussed herein above, one artifact that was considered was the possibility that some of the DMSO can decompose in the injector port of a GC/FID instrument. However, the calibration curve is capable of accounting for this thermal decomposition. For example, according to the calibration curve a level of 1 ppm dimethyl sulfide corresponds to 4.5002 area counts by our method whereas the y axis intercept (0 ppm DMS) corresponds to 1.6068 area counts. This fact confirms that an amount of DMSO is thermally decomposed to dimethyl sulfide (approximately 0.357 ppm). However, the formulator can obtain the value of dimethyl sulfide directly from the area counts obtained from a gas chromatograph. The data for the calibration curve is reproduced in tabular form in Table 2.

TABLE 2

| Area counts | DMS (ppm) |
|---|---|
| 1.58802 | 0.1 |
| 2.51464 | 0.25 |
| 3.23439 | 0.5 |
| 3.97046 | 0.75 |
| 4.24917 | 1 |

The above table is provided only as a non-limiting example of a calibration curve and the formulator can develop a similar calibration curve adapted to the specific instrument used to analyze the content of dimethyl sulfide. By using a calibration curve, high accuracy of analysis is obtainable. For example, as the level of dimethyl sulfide approaches 1 ppm or below, gas chromatograph area counts can be used, as depicted in Table 2, to determine the amount of dimethyl sulfide present in the DMSO sample.

### EXAMPLE 1

### Removal of Dimethyl Sulfide (DMS)

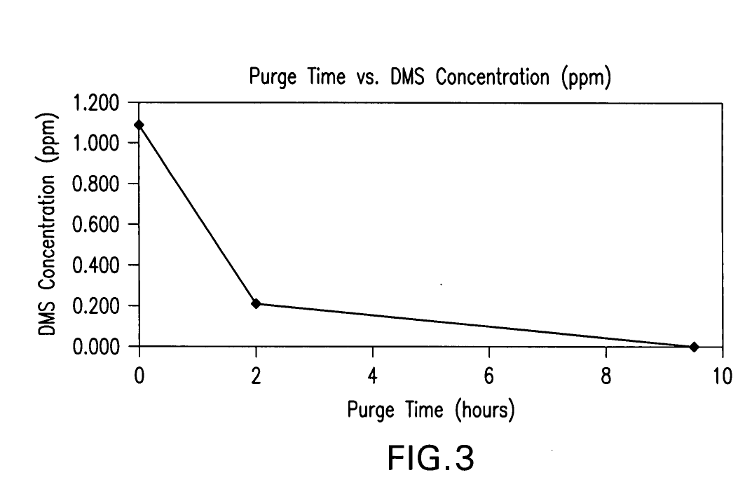

To a 55 gallon drum (500 lbs, 227 kg net weight) of dimethyl sulfoxide (DMSO) (Gaylord Chemical Pharma Solvent Grade) is heated to 45° C. over a period of about 4 hours and the DMSO maintained at this temperature. Nitrogen gas (Zero™ nitrogen by Nordon Smith Corp., Hattiesburg, Miss.) is introduced at a flow rate of 80 mL/min through a 5.08 cm internal diameter porous aluminum sparger tube charged with Rasching rings. Prior to commencing the deodorization an aliquot was drawn and analyzed by GC/FID to establish the initial concentration of dimethyl sulfide impurity. During the course of the deodorization, aliquots are withdrawn at various times during the sparging and analyzed for the content of dimethyl sulfide. Table 3 reflects the levels of dimethyl sulfide over time for this example and the data are reflected in the graph featured in FIG. 3.

TABLE 3

| Time (hr) | DMS (ppm) |
|---|---|
| 0 | 1.086 |
| 2.0 | 0.208 |
| 9.5 | ND* |

ND = not detected.

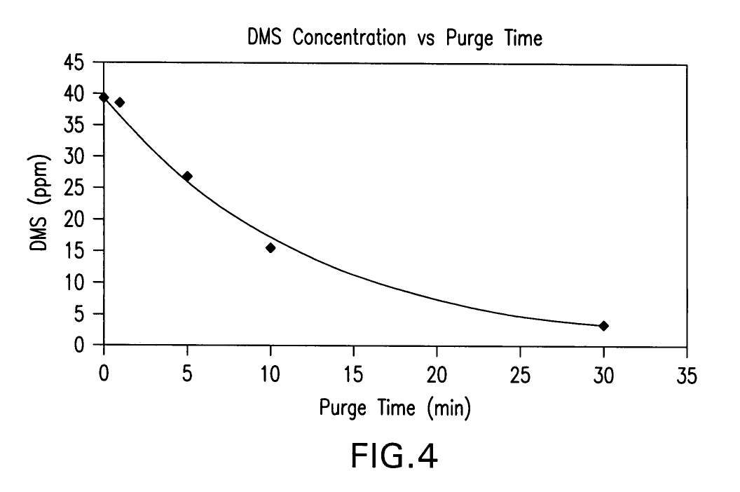

FIG. 4 depicts a typical graph of the reduction of dimethyl sulfide over time at 42.4° C. Table 4 shows the concentration of dimethyl sulfide at various times for the sample depicted in FIG. 4.

TABLE 4

| Time (min) | DMS (ppm) |
|---|---|
| 0 | 39.2 |
| 1 | 38.6 |
| 5 | 26.6 |
| 10 | 15.6 |
| 30 | 3.4 |

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A process for removing dimethyl sulfide from liquid dimethyl sulfoxide comprising:
(a) providing liquid dimethyl sulfoxide comprising dimethyl sulfide; and
(b) treating the liquid dimethyl sulfoxide with a flow of one or more gases so as to reduce the concentration of dimethyl sulfide originally present in the liquid dimethyl sulfoxide before treatment.

2. The process according to claim 1, wherein the gas is chosen from nitrogen, helium, argon, carbon dioxide, air, a C₁-C₅ alkane, and mixtures thereof.

3. The process according to claim 1, wherein the gas is introduced into the liquid dimethyl sulfoxide at a temperature of from about 18.6° C. to about 100° C.

4. The process according to claim 1, wherein the concentration of dimethyl sulfide is reduced by at least about 50% of the original concentration present in the dimethyl sulfoxide.

5. The process according to claim 1, wherein the concentration of dimethyl sulfide is reduced by at least about 99% of the original concentration present in the dimethyl sulfoxide.

6. The process according to claim 1, wherein the concentration of dimethyl sulfide present after treatment is an amount less than or equal to about 20 ppm.

7. The process according to claim 1, wherein the concentration of dimethyl sulfide present after treatment is an amount less than or equal to about 1 ppm.

8. The process according to claim 1, wherein the concentration of dimethyl sulfide present after treatment is an amount less than or equal to about 0.1 ppm.

9. The process according to claim 1, wherein after treatment the dimethyl sulfide is at a level that is undetectable.

10. The process according to claim 1, wherein the gas is introduced with a sparger.

11. The process according to claim 1, wherein the dimethyl sulfide is removed in a continuous process.

12. The process according to claim 1, wherein the dimethyl sulfide is removed in a batch process.

13. The process according to claim 1, wherein the liquid dimethyl sulfoxide has an initial purity of greater than or equal to about 96%.

14. A process for removing dimethyl sulfide from dimethyl sulfoxide, comprising the following steps, conducted in any order or simultaneously:
a) contacting liquid dimethyl sulfoxide comprising dimethyl sulfide with stream of a gas; and
b) heating the liquid dimethyl sulfoxide to a temperature of from about 30° C. to about 50° C.;
for a time sufficient to produce treated dimethyl sulfoxide having a concentration of dimethyl sulfide of less than about or equal to about 2 ppm.

15. A process for deodorizing dimethyl sulfoxide comprising:
(a) providing liquid dimethyl sulfoxide comprising one or more volatile organic compounds; and
(b) introducing into the liquid dimethyl sulfoxide a flow of one or more gases so as to reduce the concentration of one or more volatile organic compounds in the dimethyl sulfoxide by at least about 50% as compared to the concentration of the one or more volatile organic compounds originally present in the liquid dimethyl sulfoxide before treatment.

16. The process according to claim 15, wherein the gas is chosen from nitrogen, helium, argon, carbon dioxide, air, a C₁-C₅ alkane, and mixtures thereof.

17. The process according to claim 15, wherein the gas is nitrogen.

18. The process according to claim 15, wherein the gas is introduced into the liquid dimethyl sulfoxide at a temperature of from about 18.6° C. to about 100° C.

19. The process according to claim 15, wherein the concentration of the volatile organic compounds is reduced by at least about 99% of the original concentration present in the dimethyl sulfoxide.

20. The process according to claim 15, wherein the concentration of volatile organic compounds present after treatment is an amount less than or equal to about 2 ppm.

21. The process according to claim 15, wherein the liquid dimethyl sulfoxide has an initial purity of greater than or equal to about 96%.

22. The process according to claim 15, wherein the volatile organic compounds are chosen from hydrogen sulfide, dimethyl sulfide, crotyl mercaptan, ethyl mercaptan, ethyl methyl sulfide, dimethyl disulfide, or bis(methylthio)methane.

23. The process according to claim 15, wherein one of the malodorous compounds is methyl mercaptan.

24. A process for deodorizing dimethyl sulfoxide comprising (a) initially providing liquid dimethyl sulfoxide comprising one or more malodorous compounds for treatment, and (b) introducing into the liquid dimethyl sulfoxide a flow of one or more gases non-reactive with the dimethyl sulfoxide, so as to reduce the concentration of one or more of the malodorous compounds in the dimethyl sulfoxide by at least about 50% as compared to the concentration of the one or more malodorous compounds originally present in the liquid dimethyl sulfoxide before treatment.

25. A process for removing dimethyl sulfide from dimethyl sulfoxide, comprising the following steps, conducted in any order or simultaneously:
a) contacting liquid dimethyl sulfoxide comprising dimethyl sulfide with a stream of a non-reactive gas; and
b) heating the liquid dimethyl sulfoxide to a temperature of from about 30° C. to about 50° C.;
for a time sufficient to produce treated dimethyl sulfoxide having a concentration of dimethyl sulfide of less than about or equal to about 2 ppm.
